(19) United States
(12) Patent Application Publication
(10) Pub. No.: US 2024/0165188 A1
(43) Pub. Date: May 23, 2024

(54) Method for the treatment of microbial overgrowth, imbalance and infections

(71) Applicant: TRINUTRA LTD., Nes Ziona (IL)

(72) Inventors: Liki VON OPPEN-BEZALEL, Nes Ziona (IL); Mor ZEILKHA, Ramat Gan (IL)

(21) Appl. No.: 18/526,617

(22) Filed: Dec. 1, 2023

Related U.S. Application Data
(63) Continuation-in-part of application No. PCT/IL2022/050592, filed on Jun. 2, 2022.
(60) Provisional application No. 63/196,431, filed on Jun. 3, 2021.
(60) Provisional application No. 63/245,896, filed on Sep. 19, 2021.

(51) Int. Cl. A61K 36/71; A61K 31/015; A61K 31/05; A61K 31/122; A61K 31/20; A61P 17/08; A61P 31/04; A61P 31/10

(52) U.S. Cl. CPC A61K 36/71; A61K 31/015; A61K 31/05; A61K 31/122; A61K 31/20; A61P 17/08; A61P 31/04; A61P 31/10; A61K 2236/31

(57) ABSTRACT

The present invention is directed to a method for treating and/or preventing skin, mucosal and systemic conditions resulting from microbial overgrowth, imbalance or infections comprising administering a composition to a mammalian subject, wherein said composition comprises oil obtained from Nigella sativa seeds, and wherein said oil comprises thymoquinone at a concentration of at least 2.5% w/w and one or more free fatty acids (FFAs) at a concentration of 2.5% w/w or less. The present invention is also directed to dosage forms comprising the aforementioned composition.

### FIELD OF THE INVENTION

The present invention is related to the use of a composition for the treatment and prevention of skin, mucosal and systemic conditions resulting from microbial overgrowth. More specifically, the composition used in this method comprises thymoquinone and free fatty acids in particular amounts and mutual ratios.

### BACKGROUND OF THE INVENTION

The skin is the outermost tissue or organ of the body in most mammalian species, and as such, is more prone to contact with potentially harmful micro-organisms in the environment—whether by direct contact with other humans, animals or inanimate objects and surfaces, or by airborne or water borne spread.

Many different types of microbial agents may cause infection of the skin. These include bacteria, viruses, and fungal species (including yeasts). Symptoms of microbial diseases of the skin may include irritation, itching, scaling (scaly skin), redness and other signs of inflammation including swelling and the formation of blisters, vesicles and other raised lesions.

Common bacterial infections of the skin include staphylococcal infections, cellulitis, and impetigo. Acne is usually also associated with bacterial overgrowth in the affected areas of the skin. Although various different bacterial species have been associated with the development of acne vulgaris, one species, Propionibacterium acnes, is of particular significance in this regard.

Viral agents may be responsible for lesions such as shingles (caused by Herpes zoster), Herpes simplex infections and viral warts.

Fungal infections of the skin (mycoses) are especially common, particular in older subjects, or in patients having diabetes, immunosuppressed individuals, or those patients confined to bed for long periods of time.

Mycoses are found everywhere in the body. However, particularly common fungal skin infections include yeast infections (e.g., candidiasis), athlete's foot (tinea pedis), ringworm (tinea corporis) and seborrheic dermatitis. The latter condition is particularly common and is characterized by areas of skin which may be red, inflamed, scaly, greasy and itchy. Many different areas of the skin may be involved. However, in many cases, seborrheic dermatitis is seen on the scalp (dandruff), face and chest. In addition to the discomfort caused by the local symptoms, seborrheic dermatitis may also lead to problems of low esteem and related social difficulties. While several different micro-organisms may be associated with this condition, in many cases, yeast of the Malassezia genus are implicated, including M. furfur, M. globosa, M. slooffiae, M. restricta and others. Seborrhea is commonly treated with a variety of anti-fungal agents, anti-inflammatories, antihistamine agents and anti-androgens. Some of these treatment modalities have been found to be efficacious in the treatment of this condition. However, there are often many side effects and other problems associated with their use.

Another common fungal pathogen that affects the skin and mucous membranes is the yeast Candida albicans. This organism is a common cause of superficial yeast infections of both the skin and mucous membranes (particularly those of the oral cavity, vulvar region, lungs and gut). In addition, C. albicans is an opportunist pathogen that often causes systemic infection, as well as disturbances of the gastrointestinal tract, in subjects having compromised immune function (such as in AIDS, other immunodeficiencies, and following treatment with chemotherapy and steroids).

Several different plant-derived substances have previously been used in an attempt to provide a gentler solution to the problem of skin infections associated with the above-mentioned microbial organisms. However, in many cases, although these natural compositions are associated with fewer harmful effects than conventional pharmaceutical agents, they often have unacceptably low levels of efficacy.

A need therefore exists for a method of treating microbial skin infections with a herbal product, wherein said method is both much more efficacious than prior art herbal compositions and is not associated with any significant adverse effects.

The method of the present invention meets this need.

### SUMMARY OF THE INVENTION

The present inventors have unexpectedly found that compositions containing combinations of thymoquinone and free fatty acids at certain mutual ratios (i.e., relative amounts) possess significantly higher anti-microbial activity than found in prior art compositions containing these substances. In particular, compositions comprising cold-pressed seed oils (e.g., from the species Nigella sativa), wherein said oils have a thymoquinone concentration of 2.5% w/w or more, and a total concentration of free fatty acids calculated as oleic acid (AOCS Ca 5a-40) that is 2.5% or less have been found to be particularly effective. In a particularly preferred embodiments, the weight/weight concentration of thymoquinone is equal to or higher than the weight/weight concentration of free fatty acids. That is, in these embodiments, the weight ratio of thymoquinone to free fatty acids is 1:1 or numerically greater (i.e., even larger amounts of thymoquinone in relation to the amounts of the free fatty acids). In one preferred embodiment, said weight ratio of thymoquinone to free fatty acids is 1.2:1 or numerically greater.

The present invention is therefore primarily directed to a method for treating and/or preventing skin, mucosal and system conditions resulting from microbial overgrowth, imbalance or infections, comprising administering a composition a mammalian subject in need of such treatment or prevention, wherein said composition comprises oil obtained from Nigella sativa seeds, and wherein said oil comprises thymoquinone at a concentration of at least 2.5% w/w and one or more free fatty acids (FFAs) at a concentration of 2.5% w/w or less.

For the purpose of the present disclosure, the term “microbial overgrowth” is to be understood to refer to the state in which there is a disruption between the normal, healthy state of co-operation between the microbial population on the skin or mucosal surface, the host cells that form said surface, and immune system cells present on or close to said surface. Such a disruption to the normal balance between these cells (e.g., following pathogen invasion or a change in the local micro-environment, thereby favoring the growth of some microbial species over others), can lead to impaired function of the skin or mucosal surface, as well as initiate an inflammatory response.

In one embodiment, the concentration of thymoquinone in the seed oil is at least 2.5% and the concentration of FFAs is 2% or less.

In some preferred embodiments of the method of the invention, the concentration of thymoquinone in the seed oil is at least 3% w/w and the FFA concentration in the seed oil is 2.5% w/w or less.

In one specific embodiment of the method, the concentration of thymoquinone in the Nigella sativa seed oil is 3% w/w and the composition of FFA in said seed oil is 2.0% w/w.

In another preferred embodiment, the present invention encompasses a method for treating and/or preventing skin, mucosal and systemic conditions resulting from microbial overgrowth, imbalance or infections comprising administering a composition to a mammalian subject in need of such treatment or prevention, wherein said composition comprises oil obtained from Nigella sativa seeds, wherein said composition comprises thymoquinone and one or more free fatty acids (FFAs), and wherein the weight ratio of said thymoquinone to said free fatty acids is equal to or numerically greater than 1:1 (i.e. there is an even greater amount of thymoquinone in relation to the amount of FFAs). In another preferred embodiment of this aspect of the invention, the weight ratio of thymoquinone to free fatty acids is equal to or numerically greater than 1.2:1.

In another aspect, the present invention encompasses a method for treating and/or preventing acne vulgaris lesions in the skin. In a particularly preferred embodiment, the acne vulgaris lesions are associated with the presence of the bacterial organism Propionibacterium acnes, wherein said method comprises administering a composition to a mammalian subject in need of such treatment or prevention, wherein said composition comprises oil obtained from Nigella sativa seeds, wherein said composition comprises thymoquinone and one or more free fatty acids (FFAs), and wherein the weight ratio of said thymoquinone to said free fatty acids is equal to or numerically greater than 1:1 (i.e. there is an even greater amount of thymoquinone in relation to the amount of FFAs). In another preferred embodiment of this aspect of the invention, the weight ratio of thymoquinone to free fatty acids is equal to or numerically greater than 1.2:1. As in the case of the embodiments of the methods described hereinabove, the composition preferably comprises thymoquinone at a concentration of at least 2.5% w/w and one or more free fatty acids (FFAs) at a concentration of 2.5% w/w or less.

The thymoquinone and FFAs may be obtained from any suitable source, including natural materials such as plant material, including seeds, leaves, stems, roots etc., and extracts and fractions thereof. Alternatively, or additionally, synthetic versions of these compounds may be used to prepare the composition used in the present invention. In one preferred embodiment, however, the thymoquinone and FFAs are present in a cold-pressed oil obtained from the seeds of Nigella sativa. As is well known to the skilled artisan in this field, the term “cold-pressed oil” refers to oil obtained from seeds (in this case the seeds of the black cumin plant, Nigella sativa) by means of crushing seeds and forcing the natural oil out of them. This process has the advantage of not requiring the use of elevated temperature or the addition of substances not normally present in the seed, such as extraction solvents.

Generally, cold-pressed Nigella sativa oil do not contain thymoquinone and FFAs in the relative and absolute amounts as defined herein, but rather are usually characterized by having relatively (and absolutely) lower levels of thymoquinone and higher levels of FFAs. A method for producing cold-pressed Nigella sativa oil comprising the amounts and mutual ratios of thymoquinone and FFAs that are suitable for use in the method of the present invention is described in WO2019/180719. In addition to thymoquinone and FFAs, the composition used in the method of the present invention may further comprise additional active substances. In one embodiment, these additional actives may be selected from the group consisting of p-cymene, carvacrol, longifolene and nigellone.

Generally, the fatty acids most commonly present in the composition used in the method of the present invention (particularly if derived from cold-pressed Nigella sativa oil) are (C16:0) palmitic acid, oleic acid (C18:1) and linoleic acid (C18:2). However, other fatty acids, in addition to those mentioned above, or instead of them, may also be included in the composition of the present invention.

### BRIEF DESCRIPTION OF THE DRAWINGS

FIG. 1 is a bar graph depicting the size of the fungal inhibition zones caused by four different combinations of TQ and FFA, when said combinations were tested against the yeast species Malassezia furfur.

FIG. 2 is a bar graph showing the size of the fungal inhibition zones caused by five different combinations of TQ and FFA, when said combinations were tested against the yeast species Candida albicans.

FIG. 3 is a bar graph summarizing the effect of a composition of the present invention on scalp erythema in patients having seborrheic dermatitis.

FIG. 4 is a bar graph summarizing the effect of a composition of the present invention on desquamation in patients having seborrheic dermatitis.

FIG. 5 is a photograph showing the reduction in erythema and desquamation caused by treatment with a composition of the present invention for 28 days.

FIG. 6 is a bar graph summarizing the antibacterial activity of different combinations of TQ and FFA when tested against methicillin-resistant Staphylococcus aureus.

FIG. 7 is a line graph showing the antibacterial effect of different concentrations of N. sativa oil in compositions of the present invention, when tested against methicillin-resistant Staphylococcus aureus.

FIG. 8 is a bar graph summarizing and comparing the effects of a composition of the present invention and a prior art Nigella sativa oil composition on the growth of seven different microbial species that are commonly found in oily skin.

FIG. 9 is a bar graph showing and comparing the effects of a a composition of the present invention and a prior art Nigella sativa oil composition on the in vitro growth of Propionibacterium acnes.

### DETAILED DESCRIPTION OF PREFERRED EMBODIMENTS

The method of the present invention is suitable for use in the case of many type of skin, mucosal or systemic infection, including those associated (causally or otherwise) with fungi, bacteria and/or viruses. In one preferred embodiment, the method is used to treat or prevent a skin, mucosal or system infection associated with, or caused by, a fungal organism. In some preferred embodiments, the fungal organism is a yeast. The method of the present invention may be used to treat or prevent skin infections caused by, or associated with, many different yeast organisms. In some cases, the dominant yeast associated with the disease is of the Malassezia genus. In many such cases, the Malassezia yeast is a commonly occurring species such as M. furfur, M. globosa, M. slooffiae, M. restricta, and others. A very commonly found yeast in skin conditions such as seborrheic dermatitis is M. furfur. In other cases, the method of the present invention is used to treat and/or prevent overgrowth, infections and diseases associated with the opportunist pathogenic yeast Candida albicans. In some embodiments, the method of the present invention may be used to treat and/or prevent overgrowth, infections and diseases associated with fluconazole-resistant strains of C. albicans.

In another preferred embodiment, the method is used to treat or prevent a skin, mucosal or system infection associated with, or caused by, a bacterial organism. In some preferred embodiments, the bacterial organism is Staphylococcus aureus. In some particular embodiments, the bacterial organism is a methicillin-resistant Staphylococcus aureus.

In a further preferred embodiment, the method is used to treat or prevent a skin condition associated with the bacterial organisms Propionibacterium acnes. In particular, the method is to treat or prevent acne vulgaris. Thus, in this embodiment, the present invention provides a method for treating and/or preventing acne vulgaris lesions in the skin, comprising administering a composition to a mammalian subject in need of such treatment or prevention, wherein said composition comprises oil obtained from Nigella sativa seeds, and wherein said oil comprises thymoquinone at a concentration of at least 2.5% w/w, one or more free fatty acids (FFAs) at a concentration of 2.5% w/w or less, and wherein the ratio of said thymoquinone to said FFAs is equal to or greater than 1:1, preferably numerically greater than 1.2:1.

The term “associated with, or caused by” as used herein is to be understood to refer to the fact that the relevant skin, mucosal or systemic condition is characterized by an absolute and/or relative increase in the population of a certain microbial species. Thus, in some cases, there may be a proven causal relationship between this population increase and the signs and symptoms of the medical condition. In other cases, no such causal relationship can be shown. In all cases referred to herein, however, the change in microbial population on the skin or mucosal surface is co-existent with the observed pathological changes.

In one preferred embodiment of the method of the present invention, the composition is applied topically to the skin or mucosa. In this embodiment, the composition may be formulated as an oil, gel, cream, lotion, serum, conditioner or shampoo, and is applied topically to the skin or mucosa (e.g., the mucosa of the oral cavity, vulva, or rectum).

In one preferred embodiment, the concentration of Nigella sativa seed oil in the topical formulation is in the range of 0.1% w/w to 100% w/w. In another preferred embodiment, the concentration of Nigella sativa seed oil in the topical formulation is in the range of 0.1% w/w to 10% w/w. In yet another preferred embodiment, this concentration is in the range of 3% to 7% w/w.

In another preferred embodiment of the method of the present invention, the composition is administered systemically, and is preferably provided in a dosage form selected from the group consisting of drops, spray, capsules (including soft gel capsules and hard-shell capsules), tablets, caplets, beverage, bulk oil form (for oral administration using a spoon), food additive and food seasoning. The composition may also be formulated in the form of drops (e.g., for sublingual use), or incorporated into sweets, candies, jellies, nutrition bars and other confectionaries and/or beverages. Further details of the preparation of these and other formulations and dosage forms can be obtained from any standard reference on the subject, such as Remington's Pharmaceutical Sciences, Mack Publishing Co, Easton, Pa, USA, 21^{st} edition (2006).

The method of the present invention may be used to treat and/or prevent microbial infections of the skin in many different mammalian species. In one particularly preferred embodiment, however, the subject is a human being.

In another aspect, the present invention is also directed to a composition as defined hereinabove for use in the treatment and/or prevention of skin, mucosal and systemic conditions resulting from microbial overgrowth, imbalance or infections. Thus, in one embodiment, the composition used in this aspect of the invention comprises oil obtained from Nigella sativa seeds, wherein said oil comprises thymoquinone at a concentration of at least 2.5% w/w and one or more free fatty acids (FFAs) at a concentration of 2.5% w/w or less. In another embodiment, the concentration of thymoquinone in the seed oil is at least 2.5% and the concentration of FFAs is 2% or less. In a still further embodiment, the concentration of thymoquinone in the seed oil is at least 3% w/w and the FFA concentration in the seed oil is 2.5% w/w or less.

In one specific embodiment of this aspect of the invention, the concentration of thymoquinone in the Nigella sativa seed oil is 3% w/w and the composition of FFA in said seed oil is 2.0% w/w.

In another aspect, the present invention is directed to a composition for use in the treatment and/or prevention of skin, mucosal and systemic conditions resulting from microbial overgrowth, imbalance or infections, wherein said composition comprises oil obtained from Nigella sativa seeds, wherein said composition comprises thymoquinone and one or more free fatty acids (FFAs), and wherein the weight ratio of said thymoquinone to said free fatty acids is equal to or numerically greater than 1:1. In another preferred embodiment, the weight ratio of thymoquinone to free fatty acids is equal to or numerically greater than 1.2:1.

In some embodiments, the composition of the present invention is used to treat or prevent skin, mucosal and systemic conditions resulting from microbial overgrowth, imbalance or infections which are related to, or caused by, a microbial agent selected from the group consisting of fungi, bacteria and viruses.

In one embodiment of this aspect of the invention, the composition is used to treat conditions that result from microbial overgrowth, imbalance or infections caused by or related to a fungus. In one preferred embodiment, said fungus is Malassezia furfur. In one particular embodiment, the composition is used to treat seborrheic dermatitis that is associated with the presence of Malassezia furfur. In another preferred embodiment, the fungus associated with the condition being treated is Candida albicans. In some preferred embodiments, the fungus is a fluconazole-resistant strain of Candida albicans.

In another embodiment, the composition is used to treat (or prevent) conditions which result from microbial overgrowth, imbalance or infections caused by or related to a bacterial species. In one embodiment, the bacterial species is Staphylococcus aureus. In some cases, the bacterial species is a methicillin-resistant Staphylococcus aureus (MRSA).

The thymoquinone and FFAs may be obtained from any suitable source, including natural materials and/or synthetic versions of these compounds, as explained hereinabove. In one preferred embodiment, however, the thymoquinone and FFAs are present in a cold-pressed oil obtained from the seeds of Nigella sativa.

As described hereinabove, the composition used to work the present invention may further comprise additional active substances. In one embodiment, these additional actives may be selected from the group consisting of p-cymene, carvacrol, longifolene and nigellone.

In some embodiments, the composition for use in this aspect of the invention is formulated such that can be applied topically. Examples of topical formulations that may be used included (but are not limited to) those taken from the group consisting of spray, serum, cream, lotion, conditioner and shampoo. The concentration of the Nigella sativa seed oil in these topical formulations is typically in the range of 0.1%-100% (w/w). In some preferred embodiments, this concentration is in the range of 0.1% to 10% (w/w). In some further preferred embodiments, this concentration is in the range of 3% to 7% (w/w).

In other embodiments, the composition for use in this aspect of the invention is formulated such that it can be administered systemically. Dosage forms suitable for such systemic administration include (but are not limited to) those selected from the group consisting of drops, spray, capsules, tablets, caplets, beverage, food additive and food seasoning.

In a still further aspect, the present invention is directed to the use of a composition as defined hereinabove in the manufacture of a medicament for treating and/or preventing microbial infections of the skin or mucosa or for treating and/or preventing systemic infections. All of the technical features described above in relation to other aspects of the present invention also apply equally to this aspect.

In a still further aspect, the present invention is directed to a dosage form intended for topical application to the skin (e.g., the skin of the scalp, face, chest and other places in the body) or mucosa (e.g., of the mouth, vulva or rectum). Typically, this dosage form will be in the form of an oil, gel, serum, cream, lotion, conditioner or shampoo, and comprises a composition of the present invention as defined hereinabove, wherein the concentration of Nigella sativa seed oil in said dosage from is in the range of 0.1% w/w to 100% w/w, preferably 0.1% to 10% w/w and more preferably 3% to 7% w/w.

In another embodiment, the invention is directed to a dosage form intended for systemic administration, wherein said dosage form is selected from the group consisting of drops, spray, capsules, tablets, caplets, beverage, food additive and food seasoning.

Certain preferred embodiments of the invention will now be described in more detail in the following non-limiting Examples.

### EXAMPLES

### Example 1

### The In Vitro Anti-Fungal Effects of Various Combinations of Thymoquinone and Free Fatty Acids (FFAs)—Inhibition of Malassezia furfur

The aim of this in vitro study was to compare various different combinations of thymoquinone and free fatty acids with regard to their ability to inhibit the growth of the yeast species Malassezia furfur, which is commonly associated with seborrheic dermatitis, tinea versicolor and other skin and scalp conditions.

### Materials and Methods

A frozen stock of M. furfur (ATCC catalogue no. 14521) was thawed and incubated at 32 degrees Celsius for 72 hours in modified Dixon agar broth medium. Following dilution 1:10, the culture was incubated for a further 24 hours. Following further dilution, the culture is grown for an additional 3 hours prior to use.

Antibiotic assay discs (Whatman discs, supplied by Sigma-Aldrich) were prepared in advance by means of saturating them with the test solutions and controls.

Agar plates were prepared and labelled such that they each containing 6 sections, each section being used for a separate test or control substance. 150 microliters of the culture were then spread evenly over the agar surface of each plate. The prepared discs were then gently placed at the correct places on the agar surface. The plates were then incubated at 32° C. and area of inhibited fungal growth was measured and photographed every day for 2 days.

The test substances and controls used were as follows:

### Test Items

1. Black seed oil 3% thymoquinone (TQ), 2% FFA.
2. Black seed oil 3% thymoquinone (TQ), 10% FFA.
3. Black seed oil 0.5% thymoquinone (TQ), 2% FFA.
4. Black seed oil 0.5% thymoquinone (TQ), 10% FFA.

### Controls

5. Diluent medium control (100% jojoba oil)
6. Positive control (0.01% zinc pyrithione).

All concentrations given above are w/w concentrations. The various black seed oil preparations were cold-pressed Nigella sativa oils obtained from NS Oils Ltd. (Kibbutz Sa'ad, Israel).

### Results

Each of the 4 test substances was assayed for antifungal activity, as described above, at 4 different concentrations: 1%, 5%, 10% and 100%, wherein each of these concentrations (except for the 100% concentration) was obtained by diluting the black seed oil in jojoba oil. However, preliminary results showed that only the 100% concentration of each preparation displayed antifungal activity, while the control diluent (jojoba oil) enhanced the growth of M. furfur also in the presence of 1%, 5% and 10% of the test substances.

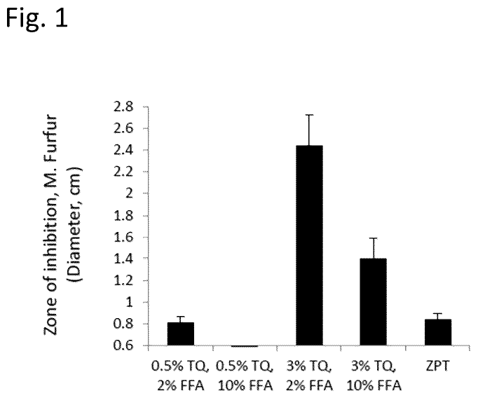

FIG. 1 is a bar graph showing the size of the fungal inhibition zones that were caused by each of the four test substances (when used at 100% concentration). The graph also shows the value obtained when the positive antifungal control's zinc pyrithione (ZPT) was used instead of a test substance. It may be seen from this graph that the composition containing 3% TQ and 2% FFA had a far greater antifungal effect than any of the other compositions tested. It should be noted that when the concentration of FFA was increased from 2% to 10%, the antifungal activity was reduced by about one half. However, the antifungal activity caused by the composition having 3% TQ and 10% FFA was still significantly greater than that seen with the ZPT positive control. When the TQ concentration was reduced to 0.5% a much lower antifungal effect was seen, even with an FFA concentration of 2%, a combination which was similar in efficacy to the positive control. Finally, the composition containing 0.5% TQ and 10% FFA appeared to be devoid of any antifungal activity.

In conclusion: this study demonstrated that unexpectedly high antifungal activity was seen with a cold-pressed black seed oil preparation in which the TQ concentration was high (3%), and the FFA concentration was low (2%). Either one of: a) reducing the TQ concentration, or b) Raising the FFA concentration reduced the antifungal activity of the preparation.

### Example 2

### The In Vitro Anti-Fungal Effects of Various Combinations of Thymoquinone and Free Fatty Acids (FFAs)—Inhibition of Candida albicans

The aim of this study was to compare various combinations of thymoquinone and free fatty acids with regard to their ability to inhibit the growth of a second fungal species—Candida albicans-growing in vitro on a solid growth medium. C. albicans is commonly associated with Infection of superficial tissues such as mucous membranes, most commonly in the mouth, lung, gut and vagina. In addition, this organism may also cause various gastrointestinal and systemic symptoms, particularly, but not only, in immunocompromised individuals (e.g., following chemotherapy, bone marrow transplantation or in subjects having an immune deficiency disease such as AIDS).

### Materials and Methods

A frozen stock of C. albicans (Robin) Berkhout (ATCC catalogue no. 96901) was thawed and incubated at 25 degrees Celsius for 72 hours in 200 YM medium. Following dilution 1:10, the culture was incubated for a further 24 hours. Following further dilution (1:10), the culture is grown for an additional 3 hours prior to use.

Antibiotic assay discs (Whatman discs, supplied by Sigma-Aldrich) were prepared in advance by means of saturating them with the test solutions and controls.

Agar plates were prepared and labelled as described in Example 1, above. The plates were then incubated at 25° C. and the area of inhibited fungal growth was measured and photographed after 24 hours.

The test substances and controls used were as follows:

### Test Items

1. Black seed oil 3% thymoquinone (TQ), 2% FFA.
2. Black seed oil 3% thymoquinone (TQ), 10% FFA.
3. Black seed oil 0.5% thymoquinone (TQ), 2% FFA.
4. Black seed oil 0.5% thymoquinone (TQ), 10% FFA.
5. Black seed oil 1.8% thymoquinone (TQ), 2% FFA formulation

### Controls

6. Negative control (empty discs)
7. Positive control I (0.05% zinc pyrithione).
8. Positive control II (100 micrograms fluconazole)

All concentrations given above are w/w concentrations. The various black seed oil preparations were cold-pressed Nigella sativa oils obtained from NS Oils Ltd. (Kibbutz Sa'ad, Israel).

### Results

Each of the 5 test substances (at a concentration of 100%, i.e., undiluted) was assayed for antifungal activity, as described above.

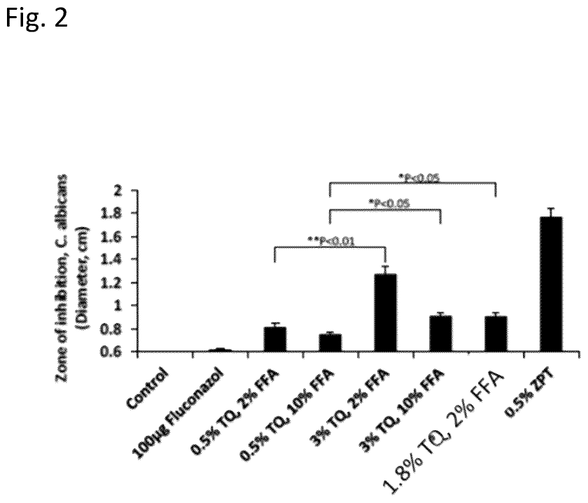

FIG. 2 is a bar graph showing the size of the fungal inhibition zones that were caused by each of the five test substances. The graph also shows the value obtained when the positive antifungal controls zinc pyrithione (ZPT) and Fluconazole were used (separately) instead of a test substance. It may be seen from this graph that the composition containing 3% TQ and 2% FFA had a far greater antifungal effect than any of the other compositions tested. It should be noted that when the concentration of FFA was increased from 2% to 10%, the antifungal activity was significantly reduced. When the TQ concentration was reduced to 0.5% a much lower antifungal effect was seen, even with an FFA concentration of 2%.

It should also be noted that the specific C. albicans strain tested is known to be fluconazole resistant, as confirmed by the lack of inhibitory activity of the fluconazole control seen in FIG. 2. The compositions of the present invention, however, inhibited the growth of this strain, indicating their utility in treating and/or preventing infection with fluconazole-resistant strains of C. albicans.

In conclusion: this study demonstrated that unexpectedly high antifungal activity against the growth of C. albicans was seen with a cold-pressed black seed oil preparation in which the TQ concentration was high (3%), and the FFA concentration was low (2%). As in the case of the anti-fungal activity directed against M. furfur (Example 1, hereinabove), either one of: a) reducing the TQ concentration, or b) Raising the FFA concentration reduced the antifungal activity of the preparation when tested against C. albicans.

### Example 3

### An Assessment of the Ability of the Method of the Present Invention to Treat Seborrheic Dermatitis in Adult Human Subjects

Ten adult subjects aged 18-65 having mild to moderate seborrheic dermatitis of the scalp were selected for this blinded, self-control study.

The test composition used in this study was ‘5% B'utyQuin® scalp serum’, supplied by Trinutra Ltd., Nes Ziona, Israel. The cosmetic serum composition contains 5% of a cold-pressed Nigella sativa seed oil containing inter alia 3% (w/w) thymoquinone and less than 2% (w/w) total free fatty acids.

Each of the subjects self-treated by means of gently massaging the scalp with the test composition until it was completed absorbed. This self-treatment was repeated, once per day, for a total of 28 days.

The subjects were asked to note, every day, any reaction to the test substance, and the degree of discomfort felt.

In addition, a clinical evaluation was performed by a dermatologist at the beginning of the study (Day 0) and at its end (Day 28). During this evaluation, both erythema and scaling (desquamation) were assessed on semiquantitative scales each with possible scores of 0, 1, 2, 3 and 4. For example, on the erythema scale, a score of 0 indicated no erythema, soothed scalp, while a score of 4 indicated severe erythema. The intermediate scores related to mild, medium and moderate levels of erythema (1, 2 and 3), respectively. A similar semi-quantitative scoring scale was used to assess the severity of scalp scaling.

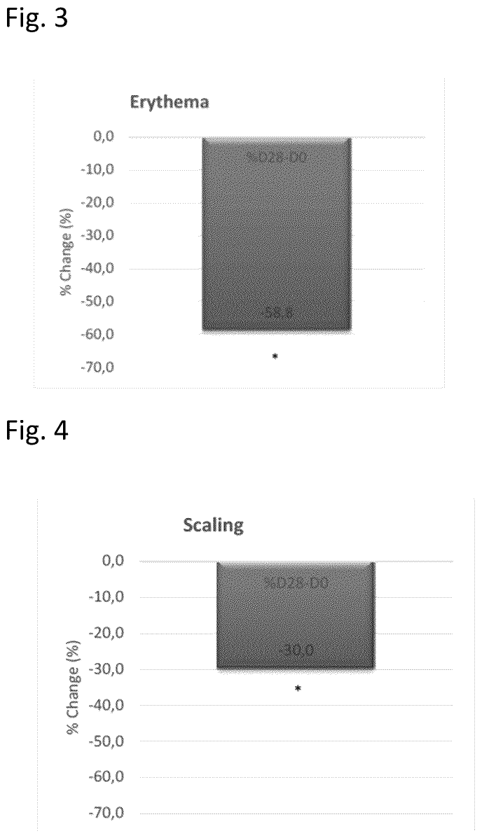

FIG. 3 indicates that the treatment caused (by day 28) a reduction of 58.8% in the severity of the scalp erythema. Similarly, a reduction of 30% in the severity of scaling was seen at day 28 (as compared with day 0), as shown in FIG. 4.

The reductions in erythema and scaling at day 28, as compared with the pre-treatment scores, were both statistically significant (p<0.05).

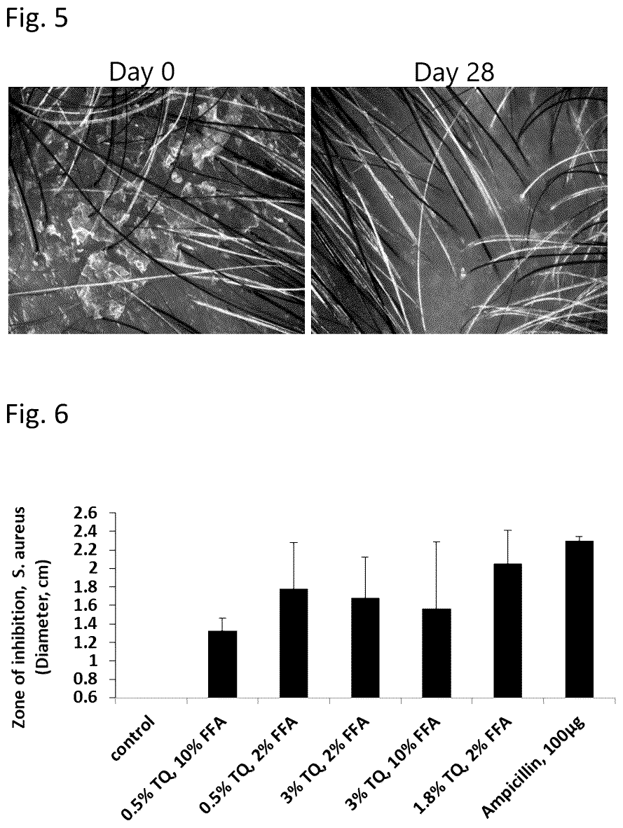

FIG. 5 provides photographic evidence of this improvement in scalp symptoms. Thus, while the left pane (pre-treatment) shows significant scale formation and scalp erythema, the situation has improved dramatically after 28 days' treatment (right pane), with almost no scaling or redness being seen.

Finally, the subjects in the study indicated (in their daily reports) that the product was highly valued for the following reasons:
Not leaving the hair feeling and looking oily.
Improved sense of comfort in the scalp
Soothing of the previously inflamed scalp

In addition, 60% of the group of subjects reported that they saw an improvement in their scalp condition over a time range extending from immediately to two weeks of use.

Furthermore, 60% felt a degree of relief in itch, scaling, dandruff and sooting of the scalp and hair during this time period.

It may be concluded from this study that the method of the present invention is highly effective in treating seborrheic dermatitis.

### Example 4

### The In Vitro Anti-Bacterial Effects of Various Combinations of Thymoquinone and Free Fatty Acids (FFAs)-Inhibition of Methicillin-Resistant Staphylococcus aureus

The aim of this study was to compare various combinations of thymoquinone and free fatty acids with regard to their ability to inhibit the growth of a bacterial species: a methicillin-resistant strain of Staphylococcus aureus.

### Introduction

The term “Methicillin-resistant Staphylococcus aureus (MRSA)” refers to a group of S. aureus that are genetically distinct from other strains of this bacterium. MRSA is responsible for some notoriously difficult-to-treat infections in humans. MRSA is particularly common in institutions such as hospitals, prisons, and nursing homes, where subjects with open wounds, invasive devices and/or weakened immune systems are at greater risk of hospital-acquired infection.

### Methods

The ability of five different N. sativa oil compositions to inhibit S. aureus growth was examined in a disc diffusion assay using a method as described in Example 1, hereinabove.

In a further study, the ability of low amounts of N. sativa oil containing 3% TQ and 2% FFAs to inhibit S. aureus was investigated using an MIC (minimum inhibitory concentration) assay. Briefly, a starter culture of S. aureus was grown in LB broth at 37° C. for 24 hours. Then, the culture was diluted to mid-log phase (0.05; 600 nm). The bacteria were incubated with black seed oil at a final volume of 200 l in U-shape 96-well plates for 5 hours and absorbance of culture was documented (600 nm). Ampicillin was used as a positive anti-bacterial control (Sigma Aldrich, 10 μg/ml).

### Statistical Analyses

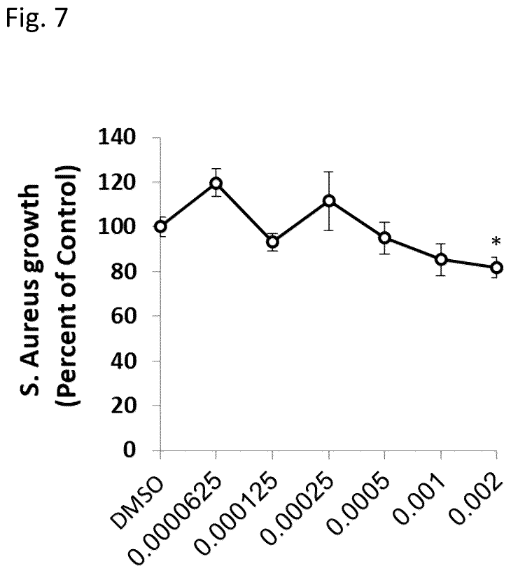

Significance analyses were calculated by student's t-test in Microsoft Excel software. The results are presented in the graphs as percentage of control (mean+/−SEM, n=3). The statistical significance each test group in comparison with the control are labeled in FIG. 7 with an asterisk (p<0.05).

### Results

The results of the disc diffusion assay are shown in FIG. 6. It is clear from these results that reduced concentrations of FFA (e.g., 2% compared with 10%) is associated with an increased antibacterial activity of the composition.

In view of the strong observed antimicrobial activity of oils rich with TQ a minimum inhibitory concentration (MIC) assay (a more sensitive antimicrobial method) was used to assess whether it is possible to reduce the oil concentration while maintaining the antimicrobial activity of N. sativa oils. The ability of black seed oil with 3% TQ and 2% FFA diluted in Dimethyl sulfoxide (DMSO, v/v) to inhibit S. aureus growth was examined following serial dilution of this oil preparation. This was achieved by first diluting the oil to 2% (v/v) in DMSO, and then further diluting in the growth medium. As seen in FIG. 7, a final concentration of 0.002% (in relation to the original oil formulation) inhibited the growth of S. aureus by almost 20%. The amount of TQ in this diluted formulation is very low: 0.00006% TQ, demonstrating a very powerful antimicrobial ability of the composition of the present invention. It can be inferred that the antibacterial properties of the composition are enhanced by its unique composition and low amounts of FFAs, enabling significant antibacterial activity even at very low concentrations in the essential oil.

### Example 5

Influence of a composition of the present invention on the in vitro growth of key microbes found in Oily Skin The aim of this study was to investigate the effect of a composition of the present invention on the in vitro growth of seven different microbial species that are commonly found in oily skin.

### Introduction

The microbial strains selected for study in this study were those predominantly found on the surface of oily skin [Byrd A. L, Belkaid, Y. and Segre J. A. (2018) “The human skin microbiome”; Nature Reviews—Microbiology], and consisted of six bacterial species and one fungal species, the details of which are listed in the following table:

| | Micro- | Strain | |
|---|---|---|---|
| | organism | collection | |
| Species | type | number | Notes |
| Corynebacterium | Bacteria | DSM44415 | Part of the commensal flora of humans. |
| simulans | | | Can be found as opportunistic pathogens |
| | | | in immunosuppressed patients. |
| Malassezia globosa | Fungus | CBS7705 | Commensal on scalp and skin. Reduced |
| | | | abundance in dandruff scalp. |
| Propionibacterium acnes | Bacteria | DSM1897 | Part of commensal skin flora and also |
| | | | involved in the pathogenesis of acne |
| | | | vulgaris. |
| Staphylococcus capitis | Bacteria | DSM20326 | Part of the commensal skin flora. Multi- |
| | | | resistant strains are difficult to treat. |
| Staphylococcus | Bacteria | DSM1798 | Colonizes the skin and mucous |
| epidermidis | | | membranes. Multi-resistant strains can |
| | | | develop. |
| Staphylococcus hominis | Bacteria | DSM20326 | Ubiquitous human skin commensal, most |
| | | | frequently isolated from healthy skin. |
| | | | Protects against opportunistic pathogens |
| | | | such as S. aureus. |
| Streptococcus mitis | Bacteria | DSM12643 | Commensal on human skin and |
| | | | oropharynx. Opportunistic pathogen in |
| | | | immunocompromised patients. |

One of these species, P. acnes is of particular interest in view of its role as a pathogenetic factor in acne vulgaris.

### Method

A liquid suspension mixture of the seven microbial species were incubated for four hours with either a 5% solution of a composition of the present invention (B'utyQuin®, comprising 3% TQ and less than 2% FFA) or a 5% solution of a non-standardized Nigella sativa oil (comprising 0.45% TQ and 9.3% FFA). In both cases, the solutions were prepared in squalane.

Prior to preparing the above-mentioned mixture, the bacterial suspensions were each adjusted to a bacteria count of approximately 103-105 CFU/ml depending on the strain. The suspensions of the individual microorganisms were mixed 1:1 (1 ml each). 500 μl of each of the test solutions (i.e., the composition of the present invention and the non-standardized Nigella sativa oil described above) were mixed with 4 ml of medium and inoculated with 500 μl of the test strain mixture. A control batch containing 500 μl of PBS instead of the product was included. Incubation took place under shaking for 4 h (at 37° C.

The bacterial counts were determined in triplicate by means of plating out the treated and control co-cultures on solid media. The plates were incubated for 24-48 h at 37° C.±2° C. (bacteria) and 5-7 d for Malassezia species at 30° C.±2° C. in an incubation chamber under the specific atmosphere for each strain.

During incubation time the colonies of the microorganisms differed in shape, color and size and were distinguishable visually, which made it possible to distinguish between the species and counting the colony forming units (CFU) for each strain. The CFU/ml and the ratios between product and control were calculated in %.

### Results

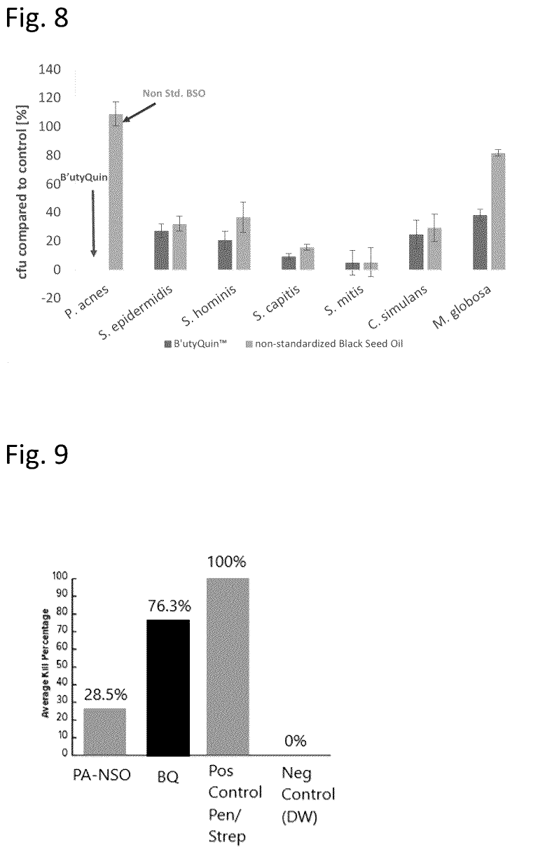

The results of this study are presented in FIG. 8, in which the first of each pair of bars represents the number of microorganisms following treatment with the composition of the present invention, while the second of each pair represents results obtained with the non-standardized N. sativa oil.

It may be seen from the graph that the composition of the present invention (B'utyQuin®) demonstrated a selective effect on microbial diversity. P. acnes was the most sensitive organism to B'utyQuin®, as it was completely absent in the culture at the end of the incubation period, while the non-standardized oil (NS-BSO) had no inhibitory effect on it.

In fact, the growth of P. Acnes was actually enhanced to 108% of the original culture count in the presence of the NS-BSO). While these results indicate that B'utyQuin® had an extremely strong antibacterial effect on P. acnes, both B'utyQuin® and the non-standardized oil had inhibitory effects on the other species in the co-culture. Thus, for example, it may be seen from FIG. 8 that B'utyQuin® reduced the growth of M. globosa by more than 60%. No significant difference between the two products was seen with regard to the growth of S. epidermidis, S. capitis, S. hominis, S. mitis and C. Simulans. It may be concluded from these results that the composition of the present invention is capable of inhibiting the growth of several key microbial species found in oily skin. In particular, said composition causes a very striking inhibition of P. acnes, an important bacterial species involved in the development of acne.

### Example 6

### Inhibition of Growth of Propionibacterium acnes In Vitro—Comparative Study

The aim of this study was to further investigate the effect of a composition of the present invention comprising 3% TQ and less than 2% FFA on the growth of the key bacterial species implicated in the pathogenesis of acne vulgaris, namely Propionibacterium acnes. The effect of this composition was compared with a non-standardized Nigella sativa oil comprising 1% TQ and 9% FFA.

### Methods

The method used in this study is the zone of inhibition method in which a monoculture of P. acnes was cultured on a solid agar plate and the compositions were applied, each separately at 100% to a filter paper disk (0.5 cm in diameter) and placed at the center of the agar plate. The compositions that were applied were: a) The composition of the present invention (B'utyQuin®, comprising 3% TQ and less than 2% FFA); b) non-standardized Nigella sativa oil (comprising 1% TQ and 9% FFA); c) a positive antibiotic control (penicillin/streptomycin); and d) a distilled water negative control. The cultured plates were incubated for 24 hours and the results of the zone of inhibition of microbial growth is measured and expressed as an average kill percentage (wherein 100% signifies total inhibition of bacterial growth by the test solutions, and 0% signifies an absence of any growth-inhibitory effect).

### Results

The results of this study are shown in FIG. 9, in which the first bar presents the results for the non-standardized N. sativa oil, the second bar relates to the composition of the present invention, the third bar is the penicillin/streptomycin positive control, and the fourth bar is the distilled water negative control.

It may be seen from this graph that while the prior art, non-standardized N. sativa oil preparation caused only a 28.5% inhibition of P. acnes growth, the composition of the present invention (second bar) had a much greater inhibitory effect (76.3%), which is closer to the 100% inhibition caused by the antibiotic positive control.

These results confirm that the composition of the present invention is highly effective in inhibiting the growth of P. acnes, and is therefore useful in the prevention, management and/or treatment of acne.

### Example 7

### Scalp Serum Formulation Containing a Composition of the Present Invention

A scalp serum formulation for use in the treatment of seborrheic dermatitis and other conditions of the scalp may be prepared by mixing together the following ingredients:

| | % w/w |
|---|---|
| Water | 72 |
| Phenoxyethanol, chlorphenesin, glycerin | 1 |
| (Microcare PHC) | |
| Propylene glycol | 5 |
| PEG-40 Hydrogenated castor oil | 1 |
| (Cremophor RH40) | |
| Cold-pressed Nigella sativa seed oil (3% | 5 |
| thymoquinone & 2.5% free fatty acids; | |
| B'utyQuin ®, supplied by Trinutra Ltd., Israel) | |
| Sodium polyacrylate, C13-14 isoparaffin, | 1 |
| Laureth-7 (Repoly 415) | |
| Alcohol | 15 |
| TOTAL: | 100 |

### Example 8

### Antiaging Cream Containing a Composition of the Present Invention

A cream formulation for use as an antiaging treatment may be prepared by mixing together the following ingredients:

| | | INGREDIENT TRADE | | |
|---|---|---|---|---|
| % | PHASE | NAME | SUPPLIER | INCI NAME |
| 68.85 | A | WATER | | Water (Aqua) |
| 0.20 | A | CARBOPOL 990 | | Carbomer |
| 3.00 | A | GLYCERIN | | Glycerine |
| 0.20 | A | DIPOTASSIUM | GFN-SELCO | Dipotassium Glycyrrhizate |
| | | GLICIRRHIZINATE | | |
| 3.00 | B | GMS SE | | Glyceryl Stearate |
| 3.50 | B | OLIVEM 1000 | | Cetearyl Olivate, Sorbitan Olivate |
| 2.00 | B | SHEA BUTTER | | Butirospermum Parkii (Shea) butter |
| 2.00 | B | CETEARYL ALCOHOL | | Cetearyl Alcohol |
| 3.00 | B | PHYTOSQUALANE | Sophim | Squalane (Olive) |
| 0.05 | B | TOCOBIOL C | BTSA | Tocopherol (mixed), Beta- |
| | | | | Sitosterol, Squalene |
| 5.00 | B | JOJOBA OIL | Jojoba Desert | Simmondsia Chinensis (Jojoba) |
| | | | | Seed Oil |
| 3.00 | B | SABODERM AB | Sabo | C12-15 Alkyl Benzoate |
| 1.50 | B | COLAFAX CPE-K | Colonial | Potassium Cetyl Phosphate |
| | | | Chemicals | |
| 1.00 | B | MICROKILL COS | Arxada (ex | Phenoxyethanol, Caprylyl Glyco, |
| | | | Lonza) | Chlorphenesine |
| 0.10 | B | BISABOLOL | GFN-SELCO | Bisabolol |
| 0.300 | C | TRIETHANOLAMINE | | Triethanolamine |
| 3.00 | D | B'UTYQUIN ® | Tri Nutra | Nigella sativa seed oil |
| 0.300 | D | FRAGRANCE | | Fragrance (Parfum) |

### Specifications

### pH: 6.0-6.8, Viscosity: 40,000-60,000 (Spindle #6, 10 Rpm @ 25° C.)

### Preparative Procedure

1. Slowly add the Carbomer to water while mixing. Allow the Carbomer to hydrate under the high shear until lump-free. Add the rest of ingredients of Phase A and heat to 75° C.
2. In a separate suitable vessel heat ingredients of Phase B to 75C, then add Phase B to the Phase A and homogenize. Start cooling and add Phase C.
3. At a temperature below 45 C add Phase D and mix.

1. A method for treating and/or preventing skin, mucosal and systemic conditions resulting from microbial overgrowth, imbalance or infections comprising administering a composition to a mammalian subject in need of such treatment or prevention, wherein said composition comprises oil obtained from Nigella sativa seeds, and wherein said oil comprises thymoquinone at a concentration of at least 2.5% w/w and one or more free fatty acids (FFAs) at a concentration of 2.5% w/w or less.

2. The method according to claim 1, wherein the concentration of thymoquinone in the Nigella sativa seed oil is at least 2.5% w/w and wherein the FFA concentration in said seed oil composition is 2.0% w/w or less.

3. The method according to claim 1, wherein the concentration of thymoquinone in the Nigella sativa seed oil is 3% w/w and the composition of FFA in said seed oil is 2.0% w/w.

4. A method for treating and/or preventing skin, mucosal and systemic conditions resulting from microbial overgrowth, imbalance or infections comprising administering a composition to a mammalian subject in need of such treatment or prevention, wherein said composition comprises oil obtained from Nigella sativa seeds, wherein said composition comprises thymoquinone and one or more free fatty acids (FFAs), and wherein the weight ratio of said thymoquinone to said free fatty acids is equal to or greater than 1:1.

5. The method according to claim 4, wherein the weight ratio of thymoquinone to free fatty acids is equal to or greater than 1.2:1.

6. The method according to claim 1, wherein the Nigella sativa seed oil is a cold-pressed seed oil.

7. The method according to claim 1, wherein the composition further comprises one or more substances selected from the group consisting of p-cymene, carvacrol, longifolene and nigellone.

8. The method according to claim 1, wherein the microbial overgrowth, imbalance or infection is related to, or caused by, a microbial agent selected from the group consisting of fungi, bacteria and viruses.

9. The method according to claim 8, wherein the microbial agent is a fungus.

10. The method according to claim 9, wherein the fungus is Malassezia furfur.

11. The method according to claim 10, wherein the condition being treated is seborrheic dermatitis.

12. The method according to claim 9, wherein the fungus is Candida albicans.

13. The method according to claim 12, wherein the fungus is a fluconazole-resistant strain of Candida albicans.

14. The method according to claim 8, wherein the microbial agent is a bacterial organism.

15. The method according to claim 14, wherein the bacterial organism is Staphylococcus aureus.

16. The method according to claim 14, wherein the bacterial organism is Propionibacterium acnes.

17. The method according to claim 1, wherein the composition is applied topically to the skin or mucosa.

18. The method according to claim 1, wherein the composition is formulated as a topical formulation selected from the group consisting of spray, serum, cream, lotion, conditioner and shampoo.

19. The method according to claim 18, wherein the concentration of Nigella sativa seed oil in the topical formulation is in the range of 0.1%-10% w/w.

20. The method according to claim 19, wherein the concentration of Nigella sativa seed oil in the topical formulation is in the range of 3%-7% w/w.

21. The method according to claim 1, wherein the composition is administered systemically, and is provided in a dosage form selected from the group consisting of drops, spray, capsules, tablets, caplets, beverage, food additive and food seasoning.

22. A method for treating and/or preventing acne vulgaris lesions in the skin, comprising administering a composition to a mammalian subject in need of such treatment or prevention, wherein said composition comprises oil obtained from Nigella sativa seeds, and wherein said oil comprises thymoquinone at a concentration of at least 2.5% w/w, one or more free fatty acids (FFAs) at a concentration of 2.5% w/w or less, and wherein the ratio of said thymoquinone to said FFAs is equal to or greater than 1:1.

23. The method according to claim 22, wherein the acne vulgaris lesions are associated with the presence of the bacterial organism Propionibacterium acnes.

24. The method according to claim 1, wherein the mammalian subject is a human subject.

25. A dosage form for topical application to the skin or mucosal membrane in the form of a serum, cream, lotion, conditioner or shampoo, wherein said dosage form comprises a composition as defined in claim 1, and wherein the concentration of Nigella sativa seed oil in said dosage form is in the range of 0.1% w/w to 100% w/w.

26. A dosage form for systemic administration, wherein said dosage form is selected from the group consisting of drops, spray, capsules, tablets, caplets, beverage, food additive and food seasoning, and wherein said dosage form comprises a composition as defined in claim 1.
